(19) United States
(12) Patent Application Publication
(10) Pub. No.: US 2013/0066185 A1
(43) Pub. Date: Mar. 14, 2013

(54) APPARATUSES, SYSTEMS AND METHODS FOR REDUCING MOTION ARTIFACTS IN BIOPOTENTIAL ELECTRODES

(71) Applicants: Trevor Kerth, La Jolla, CA (US); Yu M. Chi, La Jolla, CA (US)

(72) Inventors: Trevor Kerth, La Jolla, CA (US); Yu M. Chi, La Jolla, CA (US)

(73) Assignee: COGNIONICS, INC., San Diego, CA (US)

(21) Appl. No.: 13/609,215

(22) Filed: Sep. 10, 2012

Related U.S. Application Data
(60) Provisional application No. 61/532,337, filed on Sep. 8, 2011.

(51) Int. Cl. A61B 5/04; B05D 5/00; A61B 5/11; A61B 6/00; A61B 5/01

(52) U.S. Cl. 600/386; 600/476; 600/549; 600/595; 427/2.12

(57) ABSTRACT

An assembly for reducing motion artifacts that includes an opening on the face of the assembly, an elastic material at least partially covering the opening for suspending objects above the opening, and a sensor attached to the elastic material wherein the sensor is suspended over the opening so that the sensor may be moved in the direction of the opening so that it at least partially recesses into the opening when the sensor is positioned against a subject. The tension on the elastic material generates a force on the sensor directed towards the subject as the elastic material is stretched. Methods and systems of reducing motion artifacts including methods and systems using the assembly are also claimed.

### CROSS-REFERENCES TO RELATED PATENT APPLICATIONS

The subject matter described in the present application is related to that described in the U.S. Patent Application No. 61/532,337 to Chi and Kerth filed Sep. 8, 2011, now pending, which is incorporated by reference herein in its entirety.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND

Embodiments of the claimed subject matter relate to dry and non-contact biopotential electrode apparatuses as well as systems and methods using those apparatuses, and more particularly apparatuses as well as systems and methods that reduce electrode movement and related movement artifacts.

Recording biopotential signals such as electrocardiograms (ECG), electroencephalograms (EEGs) or electromyograms (EMGs) normally require the use of one or more conductive wet/gel electrodes fixed to the body using by an adhesive. The standard skin-adhesive electrode provides for a secure, low-resistance electrical connection between the body and the recording devices and ensures a good signal. However, the use of electrolytic gels and skin adhesives can require expensive, time consuming preparation and is often irritating and uncomfortable to the patient.

In response, dry and non-contact electrodes, which do not require gels or even direct skin contact have been recently explored as alternatives. Dry and non-contact electrodes operate by sensing the same biopotential signals but through much higher electrode impedances, since no gel is present. In the case of non-contact electrodes, they operate primarily via capacitive coupling between the subject and some insulating material (e.g., hair or clothing).

However, movement artifacts have been a crippling problem with dry and non-contact electrodes. By their nature, non-contact electrodes do not adhere to the body, allowing for relative movement, which is the largest contributor of noise. Even minute movement creates a variety of problems with the signal. Conventional designs typically use a flat smooth disc as the electrode surface that is prone to misalignment and slipping.

There are two distinct causes of these movement artifacts. The lesser arises from ‘microphonics’ where changes in the sensor-to-body displacement generate a change in voltage signal. Reducing microphonics can be accomplished by either reducing the input capacitance of the sense amplifier or by applying sufficient pressure to constrain the sensor. By far, the largest disruption occurs as the electrode ‘rubs’ against the clothing/body, generating small amounts of static charge that convert to very large voltage signals due to the high electrode impedance of the non-contact interface. Typical solutions involve using very tight harnesses or manual pressure to press the electrode to the subject's body but these solutions result in extreme pressure on the users which defeats an important purpose of non-contact electrodes (patient comfort), and even with extreme pressure the signal is still poor.

The embodiments of the claimed subject matter describe apparatuses as well as systems and methods using those apparatuses to construct, mount and utilize B non-contact electrodes that reduce the effect of motion artifacts without resorting to uncomfortable amounts of pressure.

### SUMMARY

According to one aspect of the claimed subject matter, an apparatus is provided that includes an electrode mounted on an elastic material stretched over an opening supported by a rigid face. In this embodiment, the default configuration has an electrode that extends beyond the exterior face of the assembly as it is positioned on the top surface of the elastic material. In many embodiments, when the electrode is placed on a subject, for example using a strap or other harness to support the electrode, the electrode is forced to recess into the opening, deforming the elastic material, until both the electrode and opening are approximately level on the surface of the subject. In many of the embodiments, the amount of force generated by the elastic tension is sufficient to generate tension so that the electrode is recessed into the opening. In one embodiment, a concave band behind the sensor would hold the electrode in a suitable position although with not an amount of tension to continuously make contact. The face of the assembly, which surrounds the electrode and opening, is made of a material with a high friction coefficient to prevent the assembly from sliding on the subject's body.

In many embodiments, recessing the electrode into the opening causes the elastic material to expand. This expansion causes a tension in the elastic material that provides restoring force, pushing the electrode back against the body of the subject. This restoring force is nominally independent of the tightness of the strap or harness securing the assembly, as long as enough force is applied such that the both electrode and the exterior face of the assembly conforms to the surface of the subject's body. As a result, the elastic material buffers the electrode against a subject's movements and this aids in reducing the effect of movement artifacts while at the same time avoiding the need for extremely tight and uncomfortable straps that might be uncomfortable or even painful to the subject.

To minimize the amount of slipping of misalignment between the surface of the electrode and the subject, many of the embodiments make use of a curved surface that rises above the electrode's substrate. Alternatively, in other embodiments, the electrode's surface can be made of multiple smaller curved surfaces on substrate to form bumps or any other similar deformations or formations that can aid in the gripping of the embodiments against the body of the subject. In many of the embodiments, the sensor can be positioned on the membrane or it can be in communication with the membrane rather than being attached to the membrane.

Normally, non-contact electrodes are used in the complete absence of wet gels. In very demanding applications that require low-noise signals, such as EEG, the signal from non-contact electrodes through dry hair is often too noisy to be useful. Conventional wet conductive gels are problematic because they contain skin-irritating, conductive electrolytes. Furthermore, they require careful preparation since the gel underneath each electrode must be isolated from each other to avoid an electrical short. Non-contact electrodes, which operate via capacitive coupling make it possible to utilize a non-conductive but high-dielectric constant medium that is skin friendly such as glycerin to increase the capacitive coupling between the electrode and subject without the danger of causing inter-electrode electrical shorts. Since the medium does not conduct, it can be quickly spread over the subject, the insulating material (e.g., hair) and/or the electrode. In several embodiments, a hair gel with glycerin as a major component was used. Commercially available examples of hair gels that may be used with many of the embodiments include those distributed under the brands “L.A. Looks”™ and “Wet Look”™ as these types of hair gels are optimized and well accepted for human use. Other blends consisting of skin-safe and high-dielectric constant materials may be also used with many of the embodiments.

While embodiments using the wet/gel solution using non-conductive, high dielectric constant types of media may be used with through-hair EEG elements, it may be undesirable to use those same solutions or blends for other embodiments and applications, for instance through-clothing ECG embodiments. In many of these embodiments, other methods to decrease the amount of movement noise may be used. In one specific embodiment, a method to decrease movement noise caused by static charging includes coating the insulating material, for example clothing fabric between the electrode and body, with a static electricity reducing agent.

Other embodiments may use a dryer sheet, such as a Bounce™ brand dryer sheet, for wiping the insulating material underneath the electrode. In these embodiments, using a dryer sheet to wipe the subject's clothing coats the fabric's fibers with a chemical that is lubricating and slightly conductive thus reducing the amount of static charge generation when the electrode ‘rubs’ against the fabric. The conductive properties of the chemical further improve the coupling between the electrode and the subject. Although the current embodiment uses a dryer sheet to deposit the chemicals, any number of methods are possible to deposit chemicals with similar anti-static properties (including water) on the subject and insulating material, for example clothing and hair.

According to another aspect of the claimed subject matter, an assembly is provided for reducing motion artifacts that includes an opening on the face of the assembly; an elastic material at least partially covering the opening for suspending objects above the opening; and a sensor attached to the elastic material wherein the sensor is suspended over the opening so that the sensor may be moved in the direction of the opening so that it at least partially recesses into the opening when the sensor is positioned against a subject. The tension on the elastic material generates a force on the sensor directed towards the subject as the elastic material is stretched.

According to another embodiment, the assembly includes elastic material that is comprised of a substrate joined to one or more stretchable attachments to anchor said substrate to the main body of the assembly for suspending the substrate over the opening.

According to yet another embodiment, the sensor assembly includes elastic material made up of a single sheet of stretchable material sized to at least partially cover the opening for suspending the sheet over the opening.

According to yet another embodiment, the sensor assembly includes a single sheet of stretchable material that is sized to cover the opening.

According to other embodiments of the claimed subject matter, the sensor assembly is a sensor that is a biopotential electrode, an optical sensor, a temperature sensor, a pressure sensor, a motion sensor, an acoustic sensor, a chemical sensor or any other sensor that benefits from a reduction in movement artifacts.

According to other embodiments of the claimed subject matter, the sensor assembly has a sensor which is an electrode comprised of a surface having some curvature.

According to other aspects of the claimed subject matter, the sensor assembly includes a sensor that is an electrode comprised of a plurality of curved surfaces.

According to another aspect of the claimed subject matter, an assembly is provided for with a sensor that is an electrode having a changeable thickness for varying the amount of deformation experienced by the elastic material for optimizing the amount of force applied to the subject when the sensor assembly is pressed against the subject.

According to another aspect of the claimed subject matter, the sensor in the sensor assembly may be recessed into different shaped openings. In many embodiments, the sensor is in communication with the membrane and in one specific embodiment, the sensor is resting on the membrane.

According to another embodiment, the sensor includes a horizontal component so that it can be positioned in communication with the subject at an angle. In other embodiments, the subject includes anything covering or positioned in communication with the subject. For example, a subject may include the subject's clothing or the subject's hair and/or other external coverings.

### DETAILED DESCRIPTION OF THE DRAWINGS

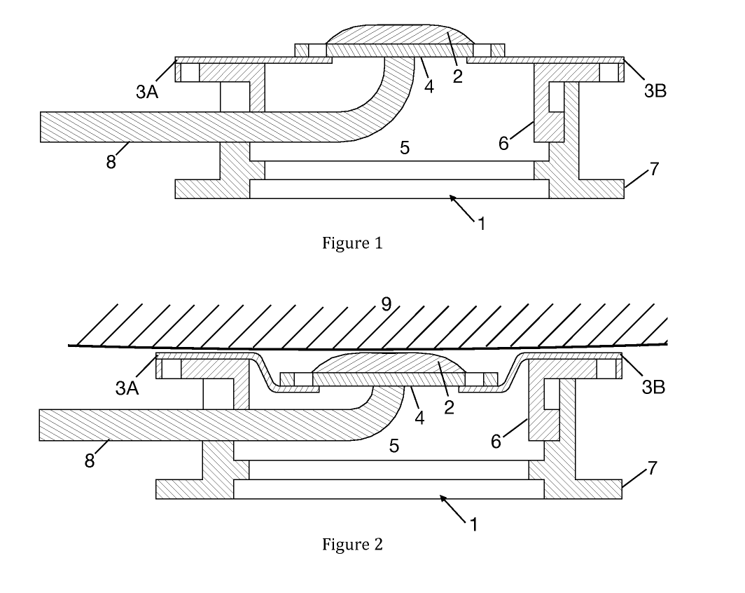

FIG. 1 is a side view of a sensor assembly embodiment showing the top plate, bottom plate, electrode, cable, opening, hollow interior, and elastic suspension system;

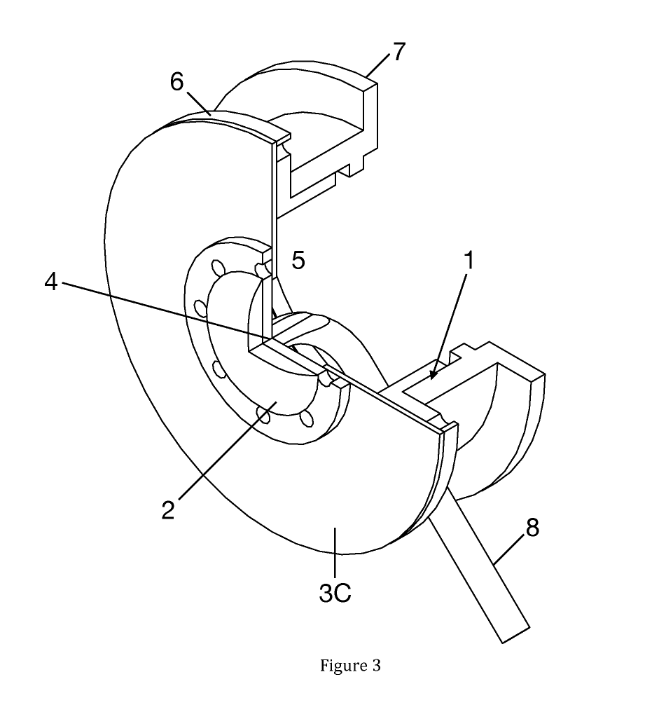

FIG. 2 is a side view of a sensor assembly embodiment pressed against the subject showing how the electrode recesses into the main housing of the sensor assembly by expanding the elastic suspension material;

FIG. 3 is an isometric cutout view of the sensor assembly embodiment of FIG. 1 showing the top plate, bottom plate, electrode, opening, hollow interior, cable and elastic suspension system;

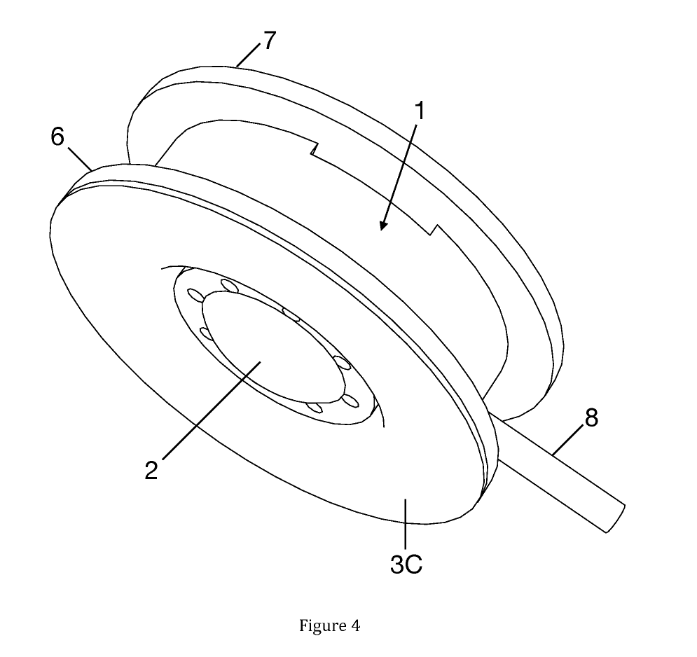

FIG. 4 is an isometric view of an embodiment showing how the electrode recesses into the assembly by expanding the elastic material;

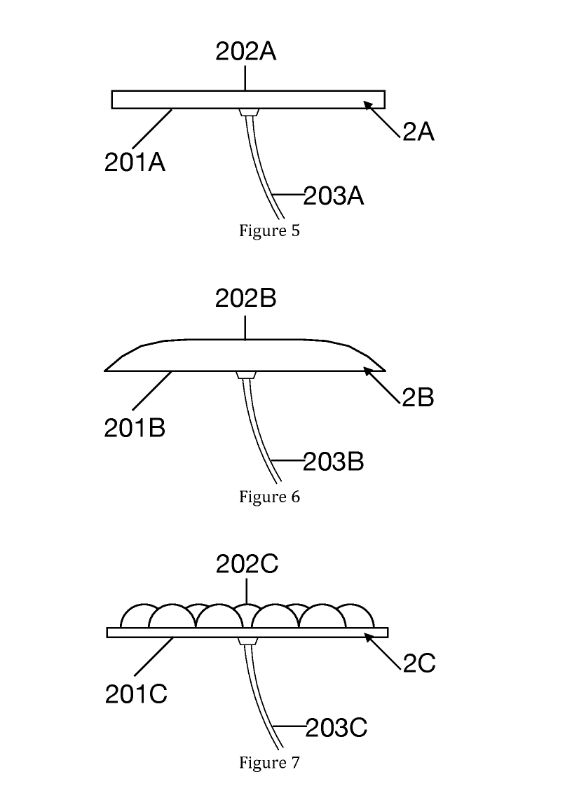

FIG. 5 is a side view of a typical flat disc electrode according to an embodiment of the claimed subject matter;

FIG. 6 is a side view of the curved electrode according to an embodiment of the claimed subject matter;

FIG. 7 is a side view of the bumped electrode according to an embodiment of the claimed subject matter; and

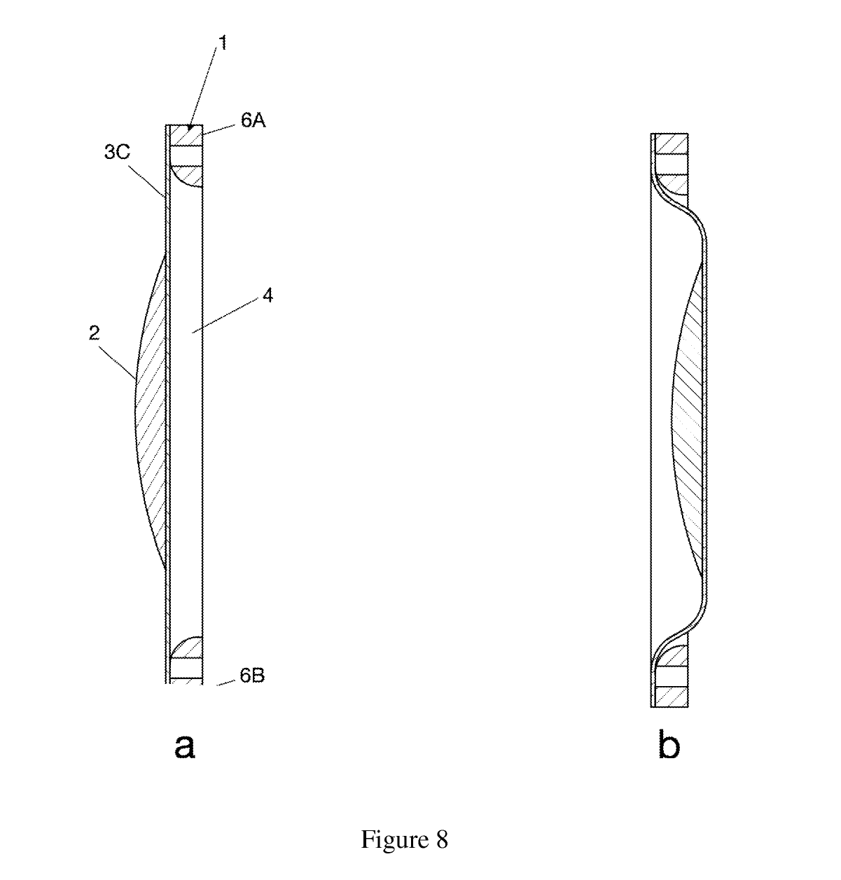

FIG. 8 is a side cutaway of another embodiment of the sensor assembly showing the same functionality without the need for the hollow interior.

### DETAILED DESCRIPTION OF THE EMBODIMENTS

According to embodiments of the claimed subject matter, various apparatuses, systems and methods systems for constructing, mounting, and utilizing dry and non-contact electrodes are provided. The apparatuses and systems of the claimed subject matter may be generally described with the reference to FIGS. 1-7 showing, but not limited to, certain exemplary embodiments of the inventive subject matter.

Turning now to the figures, FIG. 1 is a side view of an embodiment of the sensor assembly 1 designed to minimize the amount of motion on the electrode 2. The sensor assembly 1 consists of three main parts: the elastic material 3A, 3B, the top plate 6 and the base plate 7. The base plate 7 and top plate 6 are both rigid structures whereas the elastic material 3A, 3B is stretchable and provides a restoring force under deformation. The exterior face of the top plate contains an opening 4 into the hollow interior 5 in the main body the sensor assembly 1.

One embodiment contains a main body and hollow interior to fully enclose the sensor assembly. Other embodiments can dispense with the main body and hollow interior for a more compact system. Another embodiment provides the functionality of the claimed inventive subject matter using the opening, elastic material and electrode components.

The initial resting position of the elastic material 3A, 3B is flush with the exterior face of the top plate making the electrode 2 extend out from the entire sensor assembly 1. The elastic material 3A, 3B is normally a single sheet of material stretched over the exterior face of the top plate 6 but with a cut to allow for a signal cable 8 that accesses the electrode 2. The elastic material 3A, 3B can be a soft and padded material that provides a comfortable fit against the patient with no stress concentrations. In the current embodiment, the elastic material is made from spandex fabric. In many of the embodiments, any elastic material known to those skilled in the art will suffice and different materials, for example rubber, silicone or even a membrane supported by horizontal springs, can be used with the described embodiments. In these embodiments, different materials may be used to optimize the tension for different applications as desired and as known to those skilled in the art.

Several embodiments utilize a piece of spandex fabric as the elastic material 3A, 3B and covers the entire opening 4. The spandex material is stretchable which allows the electrode to recess into the sensor assembly 1 by deforming it. It is important to note, however, that the opening does not need to be entirely covered for the apparatus to function. Alternatively the elastic material 3A, 3B need not be a sheet of flexible material. As an example, the elastic material 3A, 3B can be formed using a substrate, which need not necessarily be flexible. This substrate is anchored to the top place 6 and suspended over the opening 4 by attaching springs (or any other stretchable attachments) from the substrate to the top plate 6. In this alternative embodiment, the suspension system would appear function similar to a miniaturized “trampoline.”

The electrode 2 is preferably of the active type containing a buffering amplifier to adequately sense signals from the high-impedance signal electrode. Cable 8 carries the power and signal outputs for the active circuitry near the electrode 2. Signals on cable 8 are transmitted to an instrumentation system that measures and records the signals produced by the electrode 2. In alternative embodiments, a signal cable 8 may not be necessary if the electrode 2 contained all the necessary elements to sense and wirelessly transmit data. In many of these embodiments, in order to secure the entire sensor assembly 1 to a subject such as a person, the base plate can be attached to a strap or harness system. Other embodiments may use assemblies which are made for specific subjects, for example canines or an endangered species that may have monitoring requirements.

The top plate 6 can be coated with a very high coefficient of friction to allow for the apparatus to hold onto the subject effectively. In the current embodiment, rubber strips are used. Without these rubber strips, the sensor assembly 1 would be able to slide along the subject which would exert a shear force and cause large motion and static artifacts. Alternatively, the elastic material 3A, 3B itself could be made of a high friction coefficient material such as rubber to avoid the need for separate grips.

In the embodiments shown in FIGS. 1-4, top plate 6 is secured to the base plate 7 via a bayonet lock and is completely detachable from the base plate 7. One reason the two plates may be detachable is to allow for variable shaped top plates. For smaller areas of placement or for a denser array of electrodes, a smaller diameter disk can replace the top plate. For more stability and comfort, a larger diameter disk can be used. Alternatively the top plate 6 and base plate 7 can be fused into a single unit. In the described embodiments, the rigid structures are made from plastic but they may be made from any suitable material known to those skilled in the art.

Although the described embodiments are targeted primarily for dry and non-contact biopotential sensing, it can be seen that the assembly embodiments may also be useful for reducing movement artifacts in a variety of applications. For example, the electrode 2 can be replaced with an optical sensor such as the one found in pulse oximetry. Another embodiment could involve replacing the electrode 2 with a pressure sensor, a temperature sensor, an acoustic sensor, a chemical sensor, a magnetic sensor, or any other type of sensor that is useful and for a variety of humans, animals and any moving subject.

FIG. 2 is a side view of an embodiment of the sensor assembly 1 pressed against a subject 9 showing the operation of the motion dampening properties of the embodiment. With the sensor assembly 1 pushed against the subject 9, the electrode 2 is forced to recess into the hollow interior 5 through the opening. Since the elastic material is not rigidly constraining the electrode 2, the top surface of the electrode 2 can adjust itself to lay flush against the subject 9 along with the exterior face of the top plate 6. Recessing the electrode 2 expands the elastic material 3A, 3B. The elastic modulus of the elastic material 3A, 3B is a first order constant for tension normal to the surface of the subject 9. The tension provides for an approximately constant restoring force that holds the electrode 2 against the subject 9. This force is normally constant and the elastic material 3A, 3B implements [or does it create and maintain?] a suspension system for the electrode 2 that effectively buffers the effects of subject's movement, thereby reducing the amount of artifacts. By adjusting the tightness of the elastic material 3A, 3B, the exerted tension can be modified to reach an optimal point between patient comfort and signal quality.

Instead of using an internal compression element to bias the sensor in the direction normal to the subject as shown in U.S. Patent Publication No. 2009/0030298, many of the embodiments use an elastic element 3A, 3B to evenly distribute the stress on the electrode 2. The elastic element 3A, 3B allows for increased degrees of freedom for the electrode 2 which allows for self-correcting placement and improved coupling with the subject 9. Moreover, the biasing force in the direction of the subject is not predetermined but is varied by adjusting the tightness of the elastic element 3A, 3B.

FIG. 3 shows the embodiment of FIG. 1 also in the same state as FIG. 1 but in an isometric perspective. In the embodiment shown in FIG. 3, the elastic material 3C is shown as a single sheet, with a cut near the opening 4. In this embodiment, the elastic sheet does not need to cover the entirety of the top plate 6 or the opening 4 in order to perform the motion dampening functions according to the embodiments of the inventive subject matter. The elastic material 3C may also be composed of multiple overlapping or partially overlapping layers to adjust the overall tensile properties of the embodiment.

FIG. 4 shows an embodiment in the same state as FIG. 2 in an isometric perspective with the electrode 2 recessing into the main body of the sensor assembly 1 by deforming the elastic material 3C.

FIG. 5 shows an embodiment using a known electrode design 2A which can be used as one of the possible choices for embodiments of electrode 2 shown in FIGS. 1-4. The described electrode known in the prior art consists of a substrate 201A and a conductive flat surface 202A, which may or may not be coated with an additional insulating material. A cable 203A carries signals sensed by the surface 202A into additional sensor electronics. This prior art design does not always conform well to be body and is prone to misalignment which can lead to signal loss.

FIG. 6 shows another embodiment for the electrode 2 of FIGS. 1-4. In this embodiment, the curved electrode 2B consists of a substrate 201A and a curved conductive surface 202B that extends from the substrate. The curved surface 202B better conforms to a subject than a flat shape and allows for strong coupling even if the electrode 2B is not perfectly perpendicular to the surface of the subject. The signal sensed is likewise carried on a cable 203B. Additional electronics and / or other components may be located on the electrode 2B to amplify, filter and process the signals sensed on the curved surface 202B to better reject noise and interference. The cable 203B may not be needed if the electrode 2B contained all the necessary electronics to wirelessly transmit the signals sensed on the surface 202B.

FIG. 7 shows an alternative embodiment for the electrode 2 of FIGS. 1-4 Like the previous embodiment as shown in FIG. 6, the bumped electrode 2C consists of a substrate 201C and a cable 203C to carry signals. Instead of a single curved surface of a flat disc, multiple bumps 202C are placed on the substrate 201C. The bumps 202C are made of a conductive material and act as the sensing surface. The bumped design can aid in providing a better grip against a subject while avoiding the need for the long metal fingers, which can pose an injury hazard that can sometimes be found in prior art designs. The bumps 202C can also be used to penetrate layers of hair, again avoiding the use of fingers with hard edges.

The overall height of the electrode 2, 2A, 2B, 2C can be varied to vary the amount of restoring force generated by the elastic materials 3A, 3B, 3C. A higher electrode 2 translates into more deformation in the elastic material 3A, 3B, 3C before the electrode 2 is approximately conformal against the subject 9 and the exterior face of the top plate 6. Varying the amount of restoring force generated by the elastic material 3A, 3B, 3C can be used to tune the suspension properties of the assembly 1 to optimize for patient comfort and signal quality. Assembly 1 can be made such that the electrode 2 can easily be removed and interchanged. In this way, changing the electrode 2 to change the suspension properties is likely easier than attempting to tune the elastic material 3A, 3B, 3C.

FIG. 8a is another embodiment showing an electrode assembly with reduced parts. Like the previously described embodiment, the assembly 1 suspends an electrode 2 on top of a membrane 3C. The membrane 3C is attached to the top plate 6A, 6B (shown as two pieces in the side cut-away view). An opening 4 in the top plate 6A, 6B allows the electrode 2 to recess into the opening 4 when placed against a subject that generates a tension to press the electrode 2 onto the subject with a controlled force. FIG. 8b shows the same side view but with the electrode 2 recessed into the opening 4. Unlike the previous embodiments shown in FIGS. 1-4, there is no base plate or hollow interior that reduces the profile of the assembly but retains all the key features.

Operation

In the operation of several embodiments, the sensor assembly 1 is held against the subject 9 using a strap or harness. The top plate 6 faces the subject and the harness or strap is secured on the bottom plate 7. Pressure from the harness or strap on the sensor assembly 1 causes the electrode 2 to recess into the hollow interior 5 by deforming the elastic material 3A, 3B. The restoring force generating by the elastic material 3A, 3B suspends the electrode 2 with a constant force and buffers the effects of movement, minimizing artifacts.

Electrical signals from the body and acquired by the electrode 2 are transmitted on the cable 8. In typical biopotential recording systems, an instrumentation system is connected to multiple such sensors. The difference between different sensors is amplified, processed for storage or telemetry.

Although the foregoing embodiments have been described in some detail by way of illustration and example for purposes of clarity and understanding, it will be apparent to those of ordinary skill in the art in light of the teaching of this inventive subject matter that certain changes and modifications may be made thereto without departing from the spirit or scope of the appended claims.

What is claimed is:

1. A sensor assembly for reducing motion artifacts, the assembly comprising:
(a) an opening on the face of said assembly;
(b) an elastic material at least partially covering said opening for suspending objects above said opening; and
(c) a sensor in communication with said elastic material wherein said sensor is suspended over said opening so that said sensor may be moved in the direction of said opening so that it at least partially recesses into said opening when said sensor is positioned against a subject;
(d) wherein the tension on said elastic material generates a force on said sensor directed towards said subject as said elastic material is stretched.

2. The sensor assembly of claim 1, wherein said elastic material comprises a substrate joined to one or more stretchable attachments to anchor said substrate to the main body of said assembly for suspending said substrate over said opening.

3. The sensor assembly of claim 1, wherein said elastic material comprises a single sheet of stretchable material sized to at least partially cover said opening for suspending said sheet over said opening.

4. The sensor assembly of claim 3, wherein said single sheet of stretchable material is sized to cover said opening.

5. The sensor assembly of claim 1 wherein said sensor is a biopotential electrode, an optical sensor, a temperature sensor, a pressure sensor, a motion sensor, an acoustic sensor, a chemical sensor or any other sensor that benefits from a reduction in movement artifacts.

6. The sensor assembly of claim 1 wherein said sensor is an electrode comprising of a surface having some curvature.

7. The sensor assembly of claim 1 wherein said sensor is an electrode comprised of a plurality of curved surfaces.

8. The sensor assembly of claim 1 wherein said sensor is an electrode having a changeable thickness for varying the amount of deformation experienced by said elastic material for optimizing the amount of force applied to said subject when said sensor assembly is pressed against said subject.

9. The sensor assembly of claim 1 wherein said sensor may be recessed into different shaped openings.

10. The sensor assembly of claim 1 wherein said sensor is in communication with said membrane.

11. The sensor assembly of claim 10 wherein said sensor is resting on said membrane.

12. The sensor assembly of claim 1 wherein said sensor includes a horizontal component so that it can be positioned in communication with the subject at an angle.

13. The sensor assembly of claim 1 wherein said subject further comprises anything covering or positioned in communication with said subject.

14. The sensor assembly of claim 13 wherein said covering is clothing.

15. A system of reducing artifact noise on an electrode, the system comprising coating the interface between said electrode and a subject of interest with a medium having a non conductive and high-dielectric constant property wherein said electrode's capacitive coupling to said subject is increased.

16. The system of claim 15 wherein said electrode is comprised of:
(a) an opening on the face of said assembly;
(b) an elastic material at least partially covering said opening for suspending objects above said opening; and
(c) a sensor in communication with said elastic material wherein said sensor is suspended over said opening so that said sensor may be moved in the direction of said opening so that it at least partially recesses into said opening when said sensor is positioned against a subject;
(d) wherein the tension on said elastic material generates a force on said electrode directed towards said subject as said elastic material is stretched.

17. A method for decreasing the artifact noise on an electrode assembly comprising the steps of: coating the interface between said electrode assembly and a subject of interest with a medium having a non conductive and high-dielectric constant property wherein said electrode's capacitive coupling to said subject is increased.

18. The method of claim 17 wherein said electrode assembly is comprised of:
(a) an opening on the face of said assembly;
(b) an elastic material at least partially covering said opening for suspending objects above said opening;
(c) a sensor in communication with said elastic material wherein said sensor is suspended over said opening so that said sensor may be moved in the direction of said opening so that it at least partially recesses into said opening when said sensor is positioned against a subject;
(d) wherein the tension on said elastic material generates a force on said electrode directed towards said subject as said elastic material is stretched.
